(19) United States
(12) Patent Application Publication
(10) Pub. No.: US 2023/0163006 A1
(43) Pub. Date: May 25, 2023

(54) Container Storage Facility

(71) Applicant: Daifuku Co., Ltd., Osaka-shi (JP)

(72) Inventor: Hidemoto Fukushima, Komaki-shi (JP)

(21) Appl. No.: 17/983,674

(22) Filed: Nov. 9, 2022

(30) Foreign Application Priority Data
Nov. 10, 2021 (JP) 2021-183243

(51) Int. Cl. H01L 21/673; B65G 1/06; H01L 21/677; B65G 1/04

(52) U.S. Cl. CPC H01L 21/67393; B65G 1/06; B65G 1/0407; H01L 21/67769

(57) ABSTRACT

A container storage facility including a container storage rack with a plurality of container placement sections and an inert gas supply device includes a sensor group constituted by a plurality of oxygen concentration sensors arranged in a distributed manner around the container storage rack; a diffusion fan capable of blowing air to an intended point in the container storage rack; and a controller. The controller divides an area of the container storage rack into a plurality of monitoring areas, estimates respective oxygen concentrations in the monitoring areas based on respective detection values of the oxygen concentration sensors constituting the sensor group, and, in response to any of the monitoring areas being a low oxygen concentration area in which the estimated oxygen concentration is lower than or equal to a predetermined determination threshold, operates the diffusion fan in such a manner as to blow air to the low oxygen concentration area.

### CROSS-REFERENCE TO RELATED APPLICATION

This application claims priority to Japanese Patent Application No. 2021-183243 filed Nov. 10, 2021, the disclosure of which is hereby incorporated by reference in its entirety.

### BACKGROUND OF THE INVENTION

### 1. Field of the Invention

The present invention relates to a container storage facility including a container storage rack with a plurality of container placement sections on which containers are respectively placeable, and an inert gas supply device configured to supply inert gas to each of the containers placed on the container placement sections.

### 2. Description of the Related Art

WO 2015/045582 (Patent Document 1) discloses an example of such a container storage facility. Hereinafter, in “Description of the Related Art”, the reference numerals and names in Patent Document 1 are cited in parentheses.

The purging device described in Patent Document 1 is disposed in a stocker (2) in a clean room. An internal space (6) of the stocker (2) is divided into a work area (12) and a non-work area (14). A partition (30) configured to restrict the entry of purge gas from the non-work area (14) to the work area (12) is placed at a boundary between the work area (12) and the non-work area (14). The purging device stops purging in the work area (12) when a worker enters the internal space (6). The oxygen concentration in the work area (12) is monitored, and the supply of purge gas to the non-work area (14) is also stopped in the case in which a detection result of the oxygen concentration in the work area (12) acquired from an oxygen concentration sensor (54) is lower than or equal to a predetermined value.

### SUMMARY OF THE INVENTION

The technique described in Patent Document 1 ensures workers’ safety by stopping purging of the work area when a worker enters the internal space. Moreover, if the oxygen concentration in the work area becomes lower than or equal to a predetermined value, purging of the non-work area is stopped to recover the oxygen concentration in the work area. However, the technique described in Patent

Document 1 that stops the purge gas supply and makes workers wait for the oxygen concentration to recover cannot quickly recover the oxygen concentration when the oxygen concentration decreases to be lower than or equal to a predetermined value, and, for example, if the recovery of oxygen concentration is slow, the workers may have to suspend their work. Accordingly, the technique described in Patent Document 1 may hinder the work of workers.

Therefore, it is desirable to realize a container storage facility capable of avoiding a decrease in the oxygen concentration, thereby avoiding any hindrance to the work of workers.

In view of the above, a characteristic configuration of a container storage facility is directed to a container storage facility including a container storage rack with a plurality of container placement sections on which containers are respectively placeable, and an inert gas supply device configured to supply inert gas to each of the containers placed on the container placement sections, the container storage facility including:
a sensor group constituted by a plurality of oxygen concentration sensors arranged in a distributed manner around the container storage rack;
a diffusion fan capable of blowing air to an intended point in the container storage rack; and
a controller,
wherein the controller divides an entire area of the container storage rack into a plurality of monitoring areas, estimates respective oxygen concentrations in the plurality of monitoring areas based on respective detection values of the plurality of oxygen concentration sensors constituting the sensor group, and, in response to any of the monitoring areas being a low oxygen concentration area in which the estimated oxygen concentration is lower than or equal to a predetermined determination threshold, operates the diffusion fan in such a manner as to blow air to the low oxygen concentration area.

In a container storage facility including a container storage rack with a plurality of container placement sections on which containers are respectively placeable, and an inert gas supply device configured to supply inert gas to each of the containers placed on the container placement sections, the oxygen concentration may decrease locally depending on the degree of inert gas leakage from a container. In this case, workers cannot enter the area whose oxygen concentration has decreased. According to this configuration, the oxygen concentration is monitored in each of the plurality of monitoring areas in the container storage rack, and, if there is a low oxygen concentration area in the container storage facility, it is possible to diffuse inert gas in the low oxygen concentration area by blowing air to the low oxygen concentration area. Accordingly, it is possible to avoid a local decrease in the oxygen concentration in the container storage facility, thereby avoiding any hindrance to the work of workers.

Further features and advantages of the technique according to the present disclosure will become apparent from the following description of exemplary and nonlimiting embodiments given with reference to the drawings.

### BRIEF DESCRIPTION OF THE DRAWINGS

FIG. 1 is a cross-sectional view of a container storage facility.

FIG. 2 is a view showing the positions of oxygen concentration sensors.

FIG. 3 is a view showing the positions of oxygen concentration sensors.

FIG. 4 is a view showing the positions of oxygen concentration sensors.

FIG. 5 is a view showing airflows generated by diffusion fans.

FIG. 6 is a block diagram showing functional sections according to the control of operations of diffusion fans.

FIG. 7 is a view showing monitoring areas.

FIG. 8 is a view showing an oxygen concentration map.

FIG. 9 is a chart showing a relationship between an oxygen concentration, an operation of a diffusion fan, and an operation of an exhaust fan.

FIG. 10 is a view showing another example of the container storage rack.

### DETAILED DESCRIPTION OF THE INVENTION

Hereinafter, embodiments of a container storage facility will be described with reference to the drawings. In this embodiment, a case in which the container storage facility is in a clean room will be described as an example.

### 1. Embodiment of Container Storage Facility

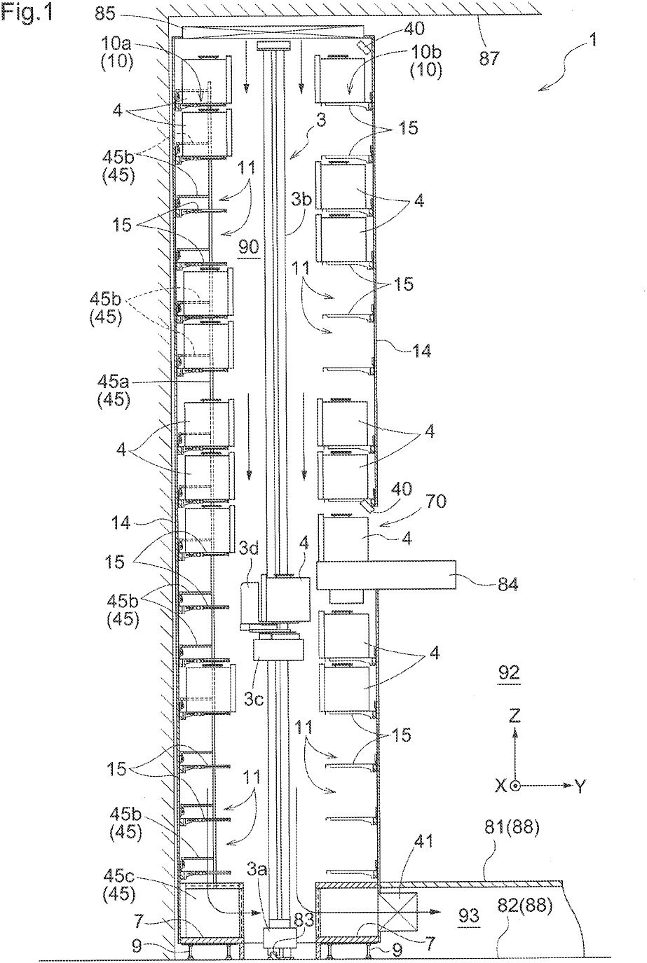

As shown in FIG. 1, a container storage facility 1 includes a container storage rack 10 and an inert gas supply device 45. The container storage rack 10 is a rack on which containers 4 are storable, and includes a plurality of container placement sections 11 on which the containers 4 are respectively placeable. The inert gas supply device 45 supplies inert gas to each of the containers 4 placed on the container placement sections 11.

In this example, as shown in FIG. 1, a specific direction along the horizontal direction (a longitudinal direction of the container storage rack 10 along the horizontal direction in this embodiment) is taken as a first direction X, a direction orthogonal to the first direction X in a vertical view along the vertical direction is taken as a second direction Y, and a direction along the vertical direction is taken as a third direction Z.

In this embodiment, each container 4 is a container that can be sealed such that the internal space is airtight. The container 4 accommodates semiconductor substrates, reticle substrates, or the like, for example. In this embodiment, the container 4 includes a body and a lid that is detachably attached to the body, and is configured such that the internal space of the container 4 is airtight when the lid is attached to the body.

In this embodiment, as shown in FIG. 1, the container storage facility 1 includes a moving device 84 configured to move the containers 4 between the inside of the container storage facility 1 and the outside of the container storage facility 1. Note that “the inside of the container storage facility 1” means the inside of a storage space 90, and “the outside of the container storage facility 1” means the outside of the storage space 90. In this embodiment, the moving device 84 is constituted by a conveyor.

The container storage facility 1 corresponds to a warehouse in which the containers 4 are storable. The container storage rack 10 is inside the container storage facility 1 including a perimeter wall 14 surrounding the container storage rack 10. The moving device 84 extends through the perimeter wall 14. That is to say, the perimeter wall 14 includes a portion with an opening 70, and the moving device 84 extends through the opening 70. The moving device 84 moves the containers 4 between an external transfer point that is outside the container storage facility 1 and an internal transfer point that is inside the container storage facility 1. The containers 4 having been moved to the external transfer point are received by an unshown external transport device. Furthermore, the moving device 84 can also receive the containers 4 from the external transport device.

In this embodiment, the container storage facility 1 is in a down-flow clean room in which clean air flows downward from the ceiling to the floor. The clean room includes a floor 88 constituted by a lower floor 82 and an upper floor 81 that is above the lower floor 82. A work space 92 is formed between the upper floor 81 and a ceiling 87. An underfloor space 93 is formed between the upper floor 81 and the lower floor 82. The upper floor 81 is structured such that air can flow therethrough. In this example, the upper floor 81 is a grating floor and has a plurality of ventilation holes that extend through the floor in the third direction Z (thickness direction). The lower floor 82 is a floor without ventilation holes, and is non-porous concrete in this example.

Air flowing from the ceiling 87 to the floor 88 passes through the upper floor 81 and further through the underfloor space 93, and flows via a connection flow path (not shown) outside the work space 92 to be supplied to the ceiling 87. Accordingly, the underfloor space 93 corresponds to an “exhaust channel”. Air supplied to the ceiling 87 is cleaned through an unshown filter, and blown downward from a discharge port (not shown) at the ceiling 87. In this way, cleaned air is circulated in the clean room.

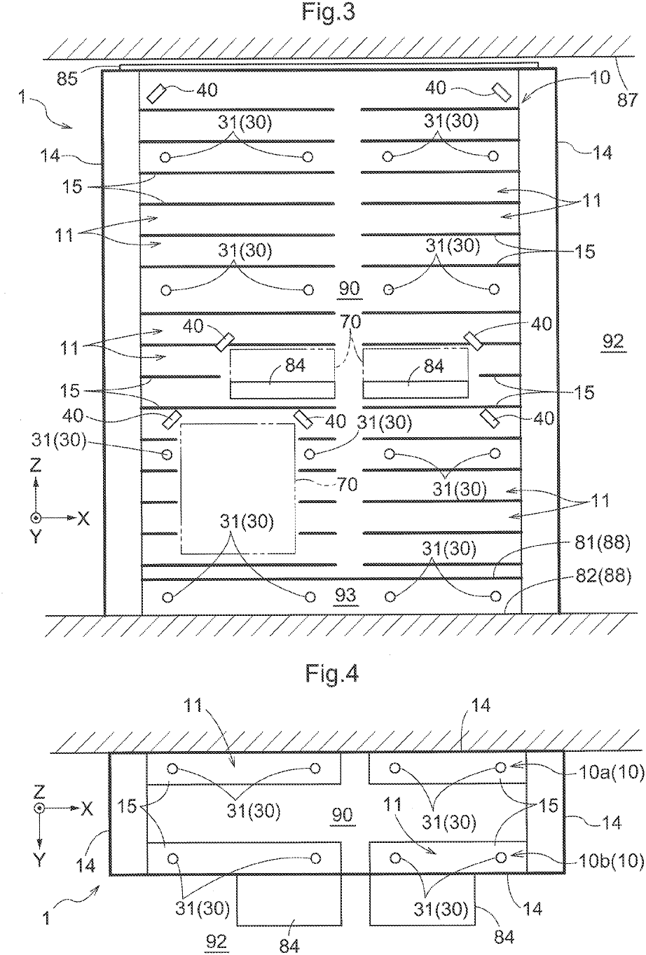

Workers in the clean room perform work (e.g., maintenance work) in the work space 92, for example, while standing on the upper floor 81. The container storage facility 1 includes a placing device (not shown) that is used by workers to store and retrieve the containers 4 into and out of the container storage facility 1. Furthermore, an opening for workers (an example of the “opening 70”) (see FIG. 3) through which workers can store and retrieve the containers 4 and an openable member (e.g., a shutter) capable of opening and closing the opening for workers are formed through the perimeter wall 14 at the position of the placing device. In this embodiment, the container storage facility 1 includes a plurality of moving devices 84 and placing devices, and these moving devices 84 and placing devices are used to store and retrieve the containers 4 into and out of the container storage facility 1.

As shown in FIG. 1, the container storage rack 10 includes the plurality of container placement sections 11 in a regular pattern. In this embodiment, the container storage facility 1 includes an internal transport device 3 configured to transport the containers 4 inside the container storage facility 1. The plurality of container placement sections 11 in the container storage rack 10 are along the first direction X. Furthermore, as shown in FIG. 1, the plurality of container placement sections 11 are not only along the first direction X but also along the third direction Z. In this embodiment, as shown in FIG. 1, the container storage rack 10 is fixed to the lower floor 82 in a state in which a bottom 7 is supported via posts 9 by the lower floor 82.

In this embodiment, the internal transport device 3 is a stacker crane, and includes a traveling body 3a that travels along a rail 83 on the floor 88, a mast 3b erected on the traveling body 3a, and a lift 3c that vertically moves along the mast 3b. The rail 83 serves as a travel path for the internal transport device 3, and is on the lower floor 82. Furthermore, in this embodiment, the rail 83 extends along the first direction X. The lift 3c includes a transfer device 3d configured to transfer the containers 4 between the lift 3c and the container placement sections 11.

The container storage rack 10 includes the plurality of container placement sections 11, and the container placement sections 11 include placing supports 15 on which the containers 4 are placeable and supportable. Specifically, the container storage rack 10 includes first accommodation racks 10a and second accommodation racks 10b facing each other with the travel path for the internal transport device 3 interposed therebetween in the second direction Y In this embodiment, as shown in FIGS. 3 and 4, two first accommodation racks 10a are side by side in the first direction X, and two second accommodation racks 10b are side by side in the first direction X.

As shown in FIG. 1, the plurality of container placement sections 11 of the container storage rack 10 are in the storage space 90. In this embodiment, the storage space 90 is a rectangular space defined by the perimeter wall 14. Furthermore, in this embodiment, the perimeter wall 14 surrounds the container storage rack 10 when the container storage facility 1 is viewed in the third direction Z. As described above, the perimeter wall 14 has the openings 70 for the moving devices 84, as well as the opening for workers (the opening 70) at the position of the placing devices.

In this embodiment, the container storage facility 1 includes a blower 85 configured to blow air from the top to the bottom of the storage space 90. The blower 85 sucks air from outside the storage space 90 and supplies it into the storage space 90. The blower 85 is disposed in such a manner as to block a rectangular opening at the upper end of the perimeter wall 14. The air blowing action of the blower 85 generates a downward airflow inside the storage space 90.

The inert gas supply device 45 supplies inert gas to each of the containers 4 placed on the container placement sections 11. The inert gas is gas that has low reactivity (does not substantially cause a problematic chemical reaction) to the contents in the containers 4, and is nitrogen gas in this embodiment. Note that the inert gas may be carbon dioxide, or a noble gas such as helium, neon, argon, krypton, xenon, or radon, instead of nitrogen gas.

In this embodiment, the inert gas supply device 45 supplies inert gas via an unshown gas supply unit to each of the containers 4 placed on the plurality of container placement sections 11. The inert gas supply device 45 includes a first pipe 45a connected to the inert gas supply source, and second pipes 45b connecting the first pipe 45a and the gas supply units. The first pipe 45a extends in the third direction Z, and the second pipes 45b branch from the first pipe 45a and extend in the second direction Y

The inert gas supply device 45 further includes a flow rate adjusting section 45c capable of adjusting the flow rate of inert gas in the first pipe 45a and the second pipes 45b. If the flow rate adjusting section 45c adjusts the flow rate of inert gas in the first pipe 45a and the second pipes 45b, the flow rate of inert gas supplied to the downstream side can also be adjusted. In FIG. 1, the first pipe 45a has the flow rate adjusting section 45c, but the second pipes 45b may have the flow rate adjusting section 45c. Inert gas from the inert gas supply device 45 is supplied to each of the containers 4, and the inert gas supplied to the containers 4 is discharged into the storage space 90. Air inside the storage space 90 containing the inert gas discharged into the storage space 90 is discharged by an exhaust fan 41 to the outside of the storage space 90. The exhaust fan 41 is in the lower space that is below the storage space 90 and does not include the container placing section 11. This generates an airflow from the inside of the container storage facility 1 to the exhaust channel.

Next, a sensor group 30 and diffusion fans 40 in the container storage facility 1 will be described. The sensor group 30 is constituted by a plurality of oxygen concentration sensors 31 arranged in a distributed manner around the container storage rack 10. The oxygen concentration sensors 31 detect the concentration of oxygen. The oxygen concentration sensors 31 may be of any type, including but not limited to zirconia type, magnetic type, semiconductor laser spectroscopy type, electrode type, and the like.

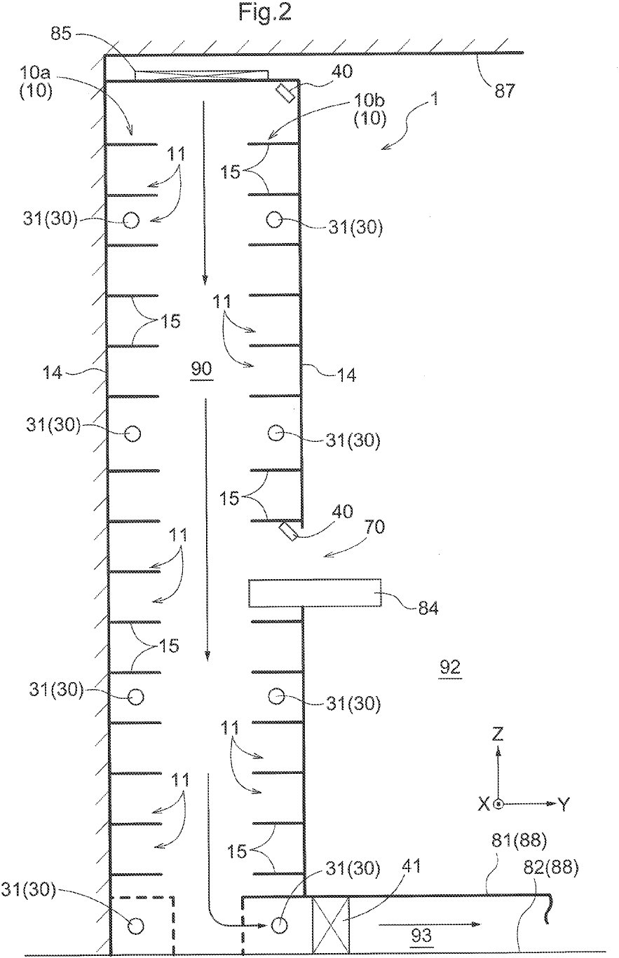

FIG. 2 is a view showing the arrangement of the oxygen concentration sensors 31 when the storage space 90 of the container storage facility 1 is viewed in the first direction X, FIG. 3 is a view showing the arrangement of the oxygen concentration sensors 31 when the storage space 90 of the container storage facility 1 is viewed in the second direction Y, and FIG. 4 is a view showing the arrangement of the oxygen concentration sensors 31 when the storage space 90 of the container storage facility 1 is viewed in the third direction Z. For facilitating the understanding, FIGS. 2, 3, and 4 do not show some of the above-described functional sections constituting the container storage facility 1.

The sensor group 30 is constituted by two or more oxygen concentration sensors 31 arranged in a distributed manner at intervals in each of the first direction X, the second direction Y, and the third direction Z. In this example, as shown in FIGS. 2 and 4, the oxygen concentration sensors 31 are at each of the first accommodation racks 10a and the second accommodation racks 10b facing each other and forming a pair constituting the container storage rack 10. Accordingly, the oxygen concentration sensors 31 are at intervals in the second direction Y Furthermore, in this example, as shown in FIGS. 3 and 4, four oxygen concentration sensors 31 are along the first direction X between a pair of surfaces of the perimeter wall 14 facing each other in the first direction X. Accordingly, in this embodiment, the oxygen concentration sensors 31 are at intervals in the first direction X. Moreover, in this example, as shown in FIGS. 2 and 3, three oxygen concentration sensors 31 are along the third direction Z between the blower 85 and the upper floor 81 facing each other in the third direction Z. Accordingly, in this embodiment, the oxygen concentration sensors 31 are at intervals in the third direction Z.

Moreover, in this embodiment, the oxygen concentration sensors 31 are at intervals in each of the first direction X and the second direction Y in the space between the height corresponding to the lower floor 82 and the height corresponding to the upper floor 81 inside the container storage rack 10, that is, in the internal space of the container storage rack 10 corresponding to the underfloor space 93. In this example, the oxygen concentration sensors 31 in the internal space of the container storage rack 10 corresponding to the underfloor space 93 are arranged in the first direction X and the second direction Y at intervals similar to those of the oxygen concentration sensors 31 in the storage space 90, and only one oxygen concentration sensor is in the third direction Z. Respective detection results of the plurality of oxygen concentration sensors 31 are transmitted to a controller 50, which will be described later.

FIGS. 2 and 3 also show the diffusion fans 40. The diffusion fans 40 are capable of blowing air to an intended point in the container storage rack 10. The intended point in the container storage rack 10 is a point at which unevenness in the oxygen concentration can be reduced through inert gas diffusion by air blown from the diffusion fans 40. Specifically, the intended point is a point at which blown air can form an airflow from one to the other of a region with a lower oxygen concentration and a region with a higher oxygen concentration, among two regions with different oxygen concentrations. Alternatively, the intended point is a point at which the flow of inert gas from the inside to the outside of the openings 70 of the container storage facility 1 can be obstructed. In order to blow air to such a point, in the example shown in FIG. 3, one or more diffusion fans 40 respectively correspond to the plurality of openings 70. Specifically, the diffusion fans 40 are obliquely above the plurality of respective openings 70 inside the perimeter wall 14, and are configured to blow air at least downward or obliquely downward. Furthermore, in the example shown in FIG. 3, the diffusion fans 40 are also at a distance from the openings 70. It is preferable that the plurality of diffusion fans 40 are arranged in a distributed manner at a distance from each other such that unevenness in the oxygen concentration inside the container storage facility 1 can be reduced.

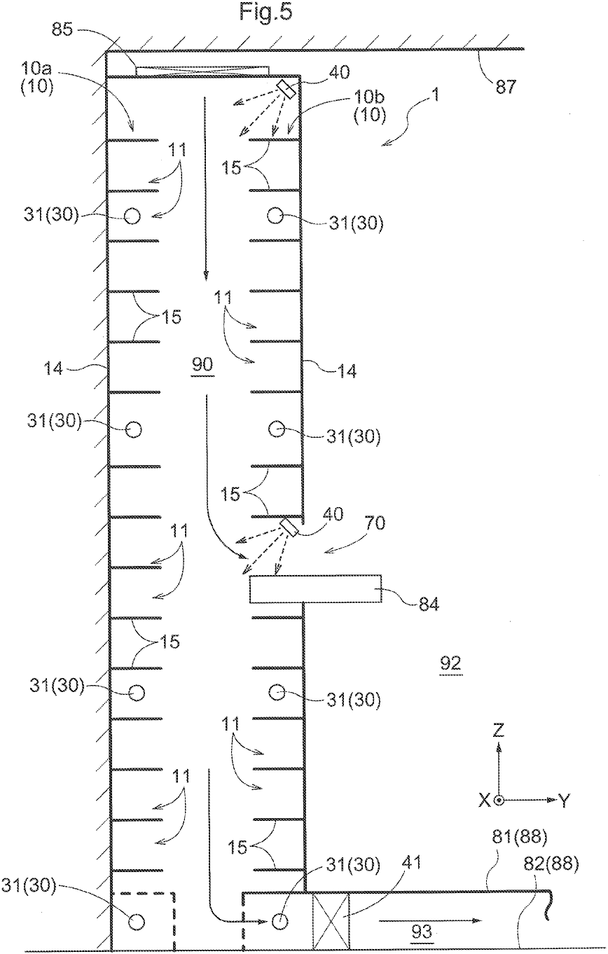

In this embodiment, as shown in FIGS. 2 and 3, the diffusion fans 40 corresponding to the openings 70 generate an airflow in a direction intersecting a direction from the inside to the outside of the openings 70, in areas adjacent to the openings 70 inside the container storage facility 1. The openings 70 inside the container storage facility 1 are the openings 70 of the perimeter wall 14, as described above. The areas adjacent to the openings 70 are areas near the openings 70 within the range in which an airflow caused in response to an operation of the diffusion fans 40 reaches. Furthermore, the direction from the inside to the outside of the openings 70 is a direction from the storage space 90 of the container storage facility 1 via the openings 70 to the work space 92. Accordingly, the direction intersecting a direction from the inside to the outside of the openings 70 corresponds to a direction that is not parallel to the direction from the storage space 90 of the container storage facility 1 via the openings 70 to the work space 92. Specifically, for example, the direction corresponds to a direction that is along the inner surface (inner wall surface) of the perimeter wall 14, a direction that is not parallel to the direction from the storage space 90 of the container storage facility 1 to the work space 92 and is from the center of the storage space 90 of the container storage facility 1 to the inner surface of the perimeter wall 14, or a direction that is not parallel to the direction from the storage space 90 of the container storage facility 1 to the work space 92 and is from the inner surface of the perimeter wall 14 to the center of the storage space 90 of the container storage facility 1. Accordingly, the diffusion fans 40 generate an airflow in a direction intersecting the direction from the storage space 90 of the container storage facility 1 via the openings 70 to the work space 92, in an area near the openings 70 of the perimeter wall 14 within the range in which an airflow caused in response to an operation of the diffusion fans 40 reaches. In FIG. 5, such airflows generated by the diffusion fans 40 are indicated by the broken lines.

The diffusion fans 40 operate in response to a command from the controller 50, which will be described later. Furthermore, it is also possible that the diffusion fans 40 are configured to be capable of changing the airflow direction in response to a command from the controller 50. The ability to change the airflow direction refers to the ability to perform a so-called oscillating movement as with an air conditioner or a fan. This makes it easier to generate an airflow in a direction intersecting a direction from the inside to the outside of the openings 70 described above. Note that the diffusion fans 40 may have a fixed airflow direction. In the case of using the diffusion fans 40 that can change airflow direction, the area in which air blown by one diffusion fan 40 reaches can be widened, and therefore, the number of diffusion fans 40 in the container storage facility 1 can be reduced.

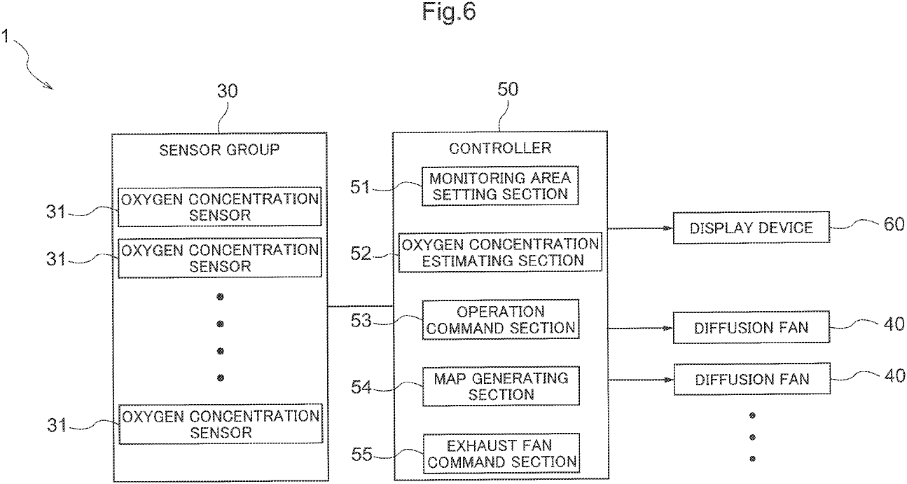

The container storage facility 1 of this embodiment includes the controller 50, and the controller 50 is configured to control an operation of the diffusion fans 40 in response to detection results of the oxygen concentration sensors 31 constituting the sensor group 30. FIG. 6 is a block diagram showing functional sections related to the controller 50 controlling operations of the diffusion fans 40.

The controller 50 includes a monitoring area setting section 51, an oxygen concentration estimating section 52, an operation command section 53, a map generating section 54, and an exhaust fan command section 55, and these functional sections are constituted by hardware or software, or both, with the CPU as the core component, in order to execute processing related to diffusion of the oxygen concentration.

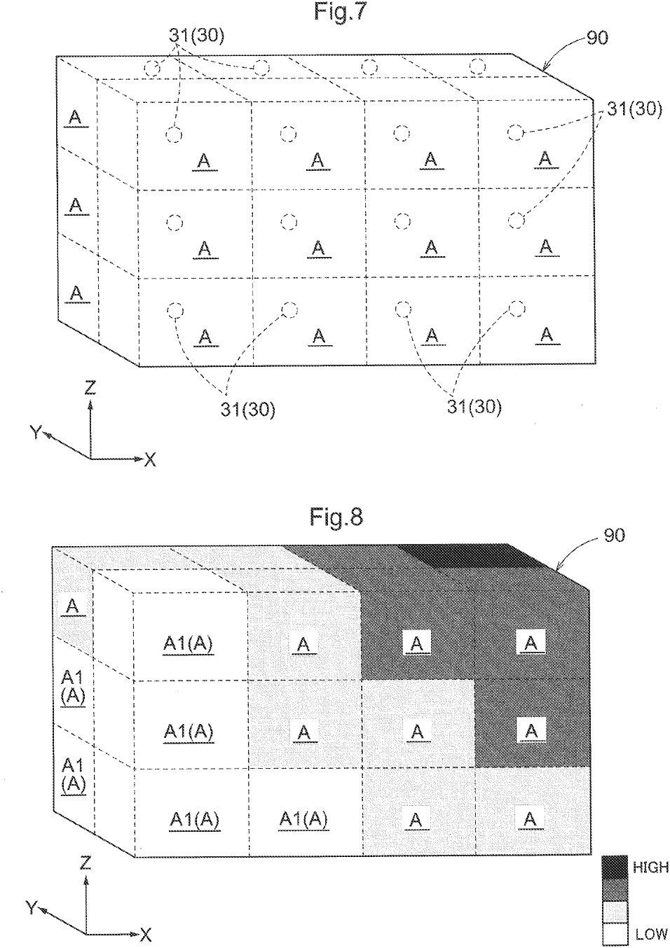

The monitoring area setting section 51 sets a plurality of monitoring areas A obtained by dividing the entire area of the container storage rack 10. In this embodiment, the entire area of the container storage rack 10 is the entire area of the storage space 90 of the container storage facility 1. “Dividing into a plurality of areas” means dividing into a plurality of areas with a prescribed size. In the example shown in FIG. 7, the storage space is divided into a plurality of monitoring areas A that are side by side in each of the first direction X, the second direction Y, and the third direction Z. The monitoring area setting section 51 divides the storage space 90 of the container storage facility 1 into a plurality of areas with a prescribed size. The divided areas are treated as the monitoring areas A. In this example, as shown in FIG. 7, the monitoring area setting section 51 divides the storage space 90 of the container storage facility 1 into four areas along the first direction X, two areas along the second direction Y, and three areas along the third direction Z. Accordingly, in the example shown in FIG. 7, 24 monitoring areas A are set. Furthermore, as shown in FIG. 7, the plurality of monitoring areas A are set such that each of the monitoring areas A contains one oxygen concentration sensor 31. However, there is no limitation to this, and, for example, there may be a monitoring area A containing an oxygen concentration sensor 31 and a monitoring area A containing no oxygen concentration sensor, or one monitoring area A may contain a plurality of oxygen concentration sensors 31.

The oxygen concentration estimating section 52 estimates the respective oxygen concentrations in the plurality of monitoring areas A, based on respective detection values of the plurality of oxygen concentration sensors 31 constituting the sensor group 30. The detection values of the oxygen concentration sensors 31 are transmitted to the oxygen concentration estimating section 52. In this example, the detection values of the oxygen concentration sensors 31 are transmitted to the oxygen concentration estimating section 52 in real time or at regular time intervals. The oxygen concentration estimating section 52 estimates the oxygen concentrations in the plurality of monitoring areas A, based on the transmitted detection values of the oxygen concentration sensors 31. In this example, each of the plurality of monitoring areas A is set to contain one oxygen concentration sensor 31, and thus the detection values of the oxygen concentration sensors 31 can be used, as they are, as estimated oxygen concentrations of the respective monitoring areas A.

In response to any of the monitoring areas A being a low oxygen concentration area A1 (see FIG. 8) in which the estimated oxygen concentration is lower than or equal to a predetermined determination threshold, the operation command section 53 operates the diffusion fans 40 in such a manner as to blow air to the low oxygen concentration area A1. The estimated oxygen concentration is the oxygen concentration in each of the monitoring areas A estimated by the oxygen concentration estimating section 52. The determination threshold is set appropriately according to the installation environment and usage conditions of the container storage rack 10. For example, the determination threshold may be set to a lower limit of oxygen concentration that allows workers in the container storage facility 1 to perform their work without any problem. The operation command section 53 determines in real time or at regular time intervals whether or not the oxygen concentration in each of the monitoring areas A estimated by the oxygen concentration estimating section 52 is lower than or equal to the determination threshold. If it is determined that the oxygen concentration is lower than or equal to the determination threshold in any monitoring area A, the operation command section 53 determines the monitoring area A as a low oxygen concentration area A1, and operates the diffusion fans 40 in such a manner as to blow air to the low oxygen concentration area A1. Blowing air to the low oxygen concentration area A1 is not limited to blowing air directly to the low oxygen concentration area A1, and also includes generating an airflow to the low oxygen concentration area A1 by blowing air to the monitoring areas A around the low oxygen concentration area A1. In any case, if the operation command section 53 operates the diffusion fans 40, it is possible to generate an airflow that diffuses inert gas in the low oxygen concentration area A1.

In this example, the operation command section 53 operates some of the plurality of diffusion fans 40 in such a manner as to blow air to the low oxygen concentration area A1. In this case, the operation command section 53 may operate all of the 40 diffusion fans individually, or may divide all of the 40 diffusion fans into a plurality of groups and operate each group individually.

Furthermore, the oxygen concentration estimating section 52 may also be configured to estimate smallest values of the respective oxygen concentrations in the plurality of monitoring areas A with use of spatial interpolation based on the detection values of all of the oxygen concentration sensors 31 constituting the sensor group 30. That is to say, for example, instead of estimating the oxygen concentration in each of the monitoring areas A, the gradient (concentration gradient) of oxygen concentration at each point is estimated with use of the detection values of the plurality of oxygen concentration sensors 31 that are adjacent to each other, and an oxygen concentration distribution in each of the plurality of monitoring areas A is estimated based on the estimated values. The oxygen concentration estimating section 52 estimates the lowest oxygen concentration in the estimated oxygen concentration distribution in each monitoring area A, as the smallest value of the oxygen concentration in the monitoring area A. This method of estimating oxygen concentration with use of spatial interpolation is applicable not only to the configuration in which each of the plurality of monitoring areas A contains one oxygen concentration sensor 31 as described above, but also to a configuration in which there are a monitoring area A containing an oxygen concentration sensor 31 and a monitoring area A containing no oxygen concentration sensor and a configuration in which one monitoring area A contains a plurality of oxygen concentration sensors 31, thereby appropriately estimating the oxygen concentration.

In the case of using such a method of estimating oxygen concentration with use of spatial interpolation, it is sufficient that the operation command section 53 determines a monitoring area A whose smallest value is lower than or equal to the determination threshold, as the low oxygen concentration area A1, and operates the diffusion fans 40 in such a manner as to blow air to the low oxygen concentration area A1.

Furthermore, if the container storage facility 1 includes a display device 60, the map generating section 54 may display, on the display device 60, an oxygen concentration map in which oxygen concentrations respectively estimated for the plurality of monitoring areas A are associated with the positions in the container storage rack 10. The oxygen concentrations respectively estimated for the plurality of monitoring areas A are the oxygen concentrations estimated by the oxygen concentration estimating section 52. Accordingly, the map generating section 54 may acquire estimation results indicating oxygen concentrations estimated by the oxygen concentration estimating section 52. The oxygen concentration map in which oxygen concentrations are associated with the positions in the container storage rack 10 is a map in which the positions of the monitoring areas A that are virtually set and the positions in the storage space 90 that is an actual space are associated with each other, wherein information indicating the estimated oxygen concentration is added to each of the regions on the map corresponding to the monitoring areas A. The map generating section 54 may generate such an oxygen concentration map and display it on the display device 60. Note that the display device 60 may be a monitor of the controller 50.

FIG. 8 shows an example of the oxygen concentration map displayed on the display device 60. It is possible to make the oxygen concentration in the storage space 90 of the container storage facility 1 easily understandable visually, by displaying such an oxygen concentration map on the display device 60 and having a worker check the map. The example shown in FIG. 8 shows the oxygen concentration in four levels, but the number of levels may be increased. Although not shown, it is also preferable to superimpose an image of the container storage rack 10 on the oxygen concentration map so that a worker checking the oxygen concentration map in FIG. 8 easily understands the map. Note that, in FIG. 8, the monitoring areas A whose oxygen concentrations are lower than or equal to a predetermined determination threshold are shown as the low oxygen concentration areas A1.

Furthermore, it is preferable that the controller 50 is configured to execute deceleration control to decelerate the airflow generated by the exhaust fan 41, in response to the oxygen concentrations in all of the plurality of monitoring areas A being higher than or equal to a deceleration threshold that is higher than or equal to the determination threshold. The deceleration threshold is set to a value that is higher than or equal to the determination threshold, appropriately according to the installation environment and usage conditions of the container storage rack 10. For example, the deceleration threshold may be set to an oxygen concentration that allows workers in the container storage facility 1 to perform their work without any problem and that does not cause problems even if air circulation in the storage space 90 is reduced. The deceleration control is control to reduce the number of rotations of the exhaust fan 41. For example, as the deceleration control, it is possible to execute feedback control to decelerate the airflow generated by the exhaust fan 41 as time passes in the case in which the oxygen concentration is higher than or equal to a deceleration threshold. Such deceleration control includes control to set the number of rotations of the exhaust fan 41 to zero, that is, stop control to stop the exhaust fan 41. The controller 50 can reduce the power consumption of the exhaust fan 41 by executing such deceleration control.

Furthermore, the controller 50 may execute deceleration prompting control to prompt a worker to perform an operation for decelerating the airflow generated by the exhaust fan 41, in response to the oxygen concentrations in all of the plurality of monitoring areas A being higher than or equal to a deceleration threshold that is higher than or equal to the determination threshold. The deceleration prompting control is control to make a notification to prompt a worker to reduce the number of rotations of the exhaust fan 41. In this example, the notification to a worker includes displaying text, graphics, or the like on the display device 60, outputting an audible message or notification sound, or the like. Such deceleration prompting control includes control to prompt a worker to set the number of rotations of the exhaust fan 41 to zero, that is, stop prompting control to prompt a worker to stop the exhaust fan 41. The controller 50 can reduce the power consumption of the exhaust fan 41 by executing such deceleration prompting control.

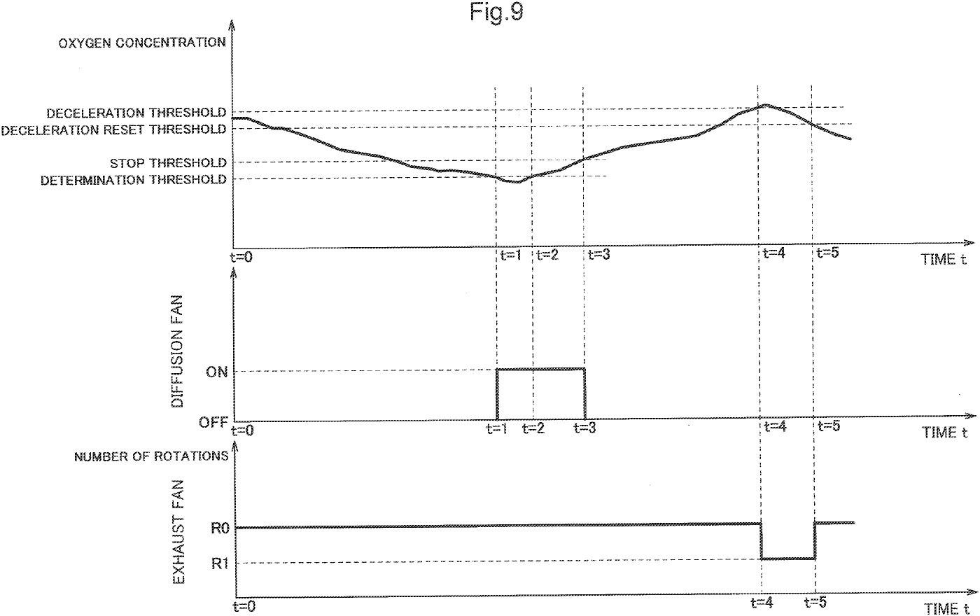

FIG. 9 shows a relationship between an oxygen concentration, an operation of the diffusion fan 40, and an operation of the exhaust fan 41. The upper chart in FIG. 9 shows a time-series change in the oxygen concentration, in which the vertical axis indicates the oxygen concentration in a given monitoring area A, and the horizontal axis indicates the time t. In the middle chart in FIG. 9, the vertical axis indicates the operation state of the diffusion fan 40, and the horizontal axis indicates the time t. In the lower chart in FIG. 9, the vertical axis indicates the number of rotations of the exhaust fan 41, and the horizontal axis indicates the time.

At t = 0, the sensor group 30 starts to measure the oxygen concentration. At this time, the diffusion fan 40 is off, and the exhaust fan 41 is driven at a predetermined number of rotations R0. At t = 1, the oxygen concentration is lower than or equal to the determination threshold. Accordingly, the controller 50 operates the diffusion fan 40 in such a manner as to blow air to the monitoring area A (the low oxygen concentration area A1) whose oxygen concentration has decreased.

Although the oxygen concentration is higher than the determination threshold at t = 2, if the diffusion fan 40 is stopped at that time, the oxygen concentration may immediately become lower than or equal to the determination threshold. In this case, the diffusion fan 40 will be operated and stopped repeatedly in a short period of time, but such repeated operation and stopping of the diffusion fan 40 in a short period of time are preferably avoided from the viewpoint of power consumption and durability of the diffusion fan 40. Therefore, it is preferable to stop the diffusion fan 40 when the oxygen concentration becomes higher than a stop threshold that is higher than the determination threshold (t=3). It will be appreciated that it is also acceptable to stop the diffusion fan 40 when the oxygen concentration becomes higher than the determination threshold at t = 2.

For example, at t = 4, if the oxygen concentration becomes higher than or equal to a deceleration threshold that is higher than or equal to the determination threshold, the above-described deceleration control or deceleration prompting control may be executed to change the number of rotations of the exhaust fan 41 from R0 to R1 that is smaller than R0. Then, for example, if the oxygen concentration becomes lower than or equal to a deceleration reset threshold that is lower than the deceleration threshold (t=5), the number of rotations of the exhaust fan 41 may be increased to R0. Alternatively, for example, if the oxygen concentration is lower than the deceleration threshold after the elapse of preset time set in advance from t = 4, the number of rotations of the exhaust fan 41 may be increased to R0.

### 2. Other Embodiments

Hereinafter, other embodiments of the container storage facility 1 will be described.

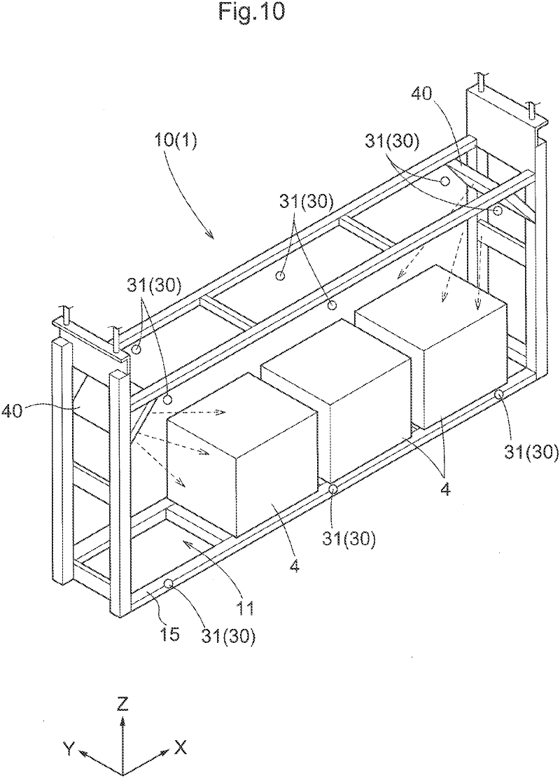

(1) In the foregoing embodiment, the case in which the container storage facility 1 is configured such that the perimeter wall 14 surrounds the container storage rack 10, that is, the storage space 90 is a space sealed by the perimeter wall 14 was described as an example. However, there is no limitation to such a configuration, and, for example, the container storage rack 10 may have a structure open to the surroundings as in a buffer (STB) shown in FIG. 10. Furthermore, a plurality of buffers shown in FIG. 10 may be in the first direction X, the second direction Y, and the third direction Z. In this case as well, the container storage facility 1 includes the sensor group 30 constituted by the plurality of oxygen concentration sensors 31 arranged in a distributed manner around the container storage rack 10, and one or more diffusion fans 40 capable of blowing air to an intended point in the container storage rack 10. In the example shown in the drawing, the sensor group 30 includes two or more oxygen concentration sensors 31 arranged in a distributed manner at intervals in each of the first direction X, the second direction Y, and the third direction Z in such a manner as to surround the container storage rack 10. Furthermore, a plurality of diffusion fans 40 (two diffusion fans, in the example shown in the drawing) are arranged in a distributed manner in the arrangement direction of the container placement sections 11. In this example, the diffusion fans 40 are arranged in such a manner as to blow air downward from the upper side to the container placement sections 11.

(2) In the foregoing embodiment, the case in which the container storage facility 1 includes a plurality of diffusion fans 40 was described as an example. However, embodiments of the container storage facility 1 are not limited to this sort of configuration. The number of diffusion fans 40 in the container storage facility 1 may be one.

(3) In the foregoing embodiment, the display device 60 was described as a monitor of the controller 50. However, embodiments of the container storage facility 1 are not limited to this sort of configuration. The display device 60 may be a monitor of a mobile terminal held by a worker, or smart glasses (a display device integrated with glasses) if the worker is wearing smart glasses.

(4) In the foregoing embodiment, the controller 50 was described as including the monitoring area setting section 51, the oxygen concentration estimating section 52, the operation command section 53, the map generating section 54, and the exhaust fan command section 55. However, embodiments of the container storage facility 1 are not limited to this sort of configuration. The functional sections constituting the controller 50 were described as an example, and the way in which the functional sections are divided may be changed as needed. It is also possible to configure the controller 50 to have other functional sections.

(5) In the foregoing embodiment, the configuration in which the entire area of the container storage rack 10 is divided into a plurality of monitoring areas A that are side by side in each of the first direction X, the second direction Y, and the third direction Z was described as an example, but there is no limitation to this. For example, the entire area of the container storage rack 10 may be divided into a plurality of areas in any one of the first direction X, the second direction Y, and the third direction Z. Alternatively, the entire area of the container storage rack 10 may be divided into a plurality of areas in any two of the first direction X, the second direction Y, and the third direction Z, and not divided in the remaining one direction.

(6) In the foregoing embodiment, the configuration in which the container storage facility 1 includes the diffusion fans 40 corresponding to the openings 70 of the perimeter wall 14, and the diffusion fans 40 at a distance from the openings 70 was described as an example, but there is no limitation to this. For example, the container storage facility 1 may include only the diffusion fans 40 corresponding to the openings 70 of the perimeter wall 14. Alternatively, the container storage facility 1 may include only the diffusion fans 40 at a distance from the openings 70.

(7) Note that the configurations disclosed in the foregoing embodiments can be applied in combination with configurations disclosed in other embodiments as long as no contradiction arises. With respect to other configurations, the embodiments disclosed herein are merely exemplary in all respects. Therefore, various modifications can be appropriately made without departing from the gist of the present disclosure.

### 3. Summary of Embodiments Above

Hereinafter, a summary of the container storage facility described above will be described.

A container storage facility is a container storage facility including a container storage rack with a plurality of container placement sections on which containers are respectively placeable, and an inert gas supply device configured to supply inert gas to each of the containers placed on the container placement sections, the container storage facility including:
a sensor group constituted by a plurality of oxygen concentration sensors arranged in a distributed manner around the container storage rack;
a diffusion fan capable of blowing air to an intended point in the container storage rack; and
a controller,
wherein the controller divides an entire area of the container storage rack into a plurality of monitoring areas, estimates respective oxygen concentrations in the plurality of monitoring areas based on respective detection values of the plurality of oxygen concentration sensors constituting the sensor group, and, in response to any of the monitoring areas being a low oxygen concentration area in which the estimated oxygen concentration is lower than or equal to a predetermined determination threshold, operates the diffusion fan in such a manner as to blow air to the low oxygen concentration area.

In a container storage facility including a container storage rack with a plurality of container placement sections on which containers are respectively placeable, and an inert gas supply device configured to supply inert gas to each of the containers placed on the container placement sections, the oxygen concentration may decrease locally depending on the degree of inert gas leakage from a container. In this case, workers cannot enter the area whose oxygen concentration has decreased. According to this configuration, the oxygen concentration is monitored in each of the plurality of monitoring areas in the container storage rack, and, if there is a low oxygen concentration area in the container storage facility, it is possible to diffuse inert gas in the low oxygen concentration area by blowing air to the low oxygen concentration area. Accordingly, it is possible to avoid a local decrease in the oxygen concentration in the container storage facility, thereby avoiding any hindrance to the work of workers.

In this example, it is preferable that the sensor group includes two or more of the oxygen concentration sensors arranged in a distributed manner at intervals in each of a first direction, a second direction, and a third direction, the first direction being a specific direction along a horizontal direction, the second direction being a direction orthogonal to the first direction in a vertical view along a vertical direction, and the third direction being a direction along the vertical direction, and

the controller estimates smallest values of respective oxygen concentrations in the plurality of monitoring areas with use of spatial interpolation based on detection values of all of the oxygen concentration sensors constituting the sensor group, and determines a monitoring area whose smallest value is lower than or equal to the determination threshold, as the low oxygen concentration area.

According to this configuration, even in the case in which the number of oxygen concentration sensors arranged in a distributed manner around the container storage rack is relatively small, whether or not there is a low oxygen concentration area can be precisely determined.

Furthermore, it is preferable that the diffusion fan is configured to be capable of changing an airflow direction in response to a command from the controller.

According to this configuration, even in the case in which the number of diffusion fans in the container storage facility is relatively small, air can be blown to a plurality of monitoring areas. Furthermore, according to this configuration, the number of diffusion fans in the container storage facility can be reduced, and thus it is easy to reduce the cost compared with the case with a large number of diffusion fans.

Furthermore, it is preferable that the container storage facility further includes a display device, and

the controller displays, on the display device, an oxygen concentration map in which oxygen concentrations respectively estimated for the plurality of monitoring areas are associated with positions in the container storage rack.

According to this configuration, oxygen concentrations are respectively estimated for a plurality of monitoring areas obtained by dividing the entire area of the container storage rack, and the estimation result is displayed as an oxygen concentration map on the display device, and thus it is easy for workers to understand the oxygen concentration at each point in the container storage rack.

Furthermore, it is preferable that the container storage rack is inside a warehouse including a perimeter wall surrounding the container storage rack,
the perimeter wall includes a portion with an opening, and
the diffusion fan generates an airflow in a direction intersecting a direction from an inside to an outside of the opening, in an area adjacent to the opening inside the warehouse.

According to this configuration, air with a low oxygen concentration can be diffused inside the opening of the perimeter wall, and thus air with a low oxygen concentration can be prevented from flowing to the outside from the opening of the perimeter wall.

Furthermore, it is preferable that the container storage rack is inside a warehouse including a perimeter wall surrounding the container storage rack, and

the container storage facility further includes an exhaust fan configured to generate an airflow from an inside of the warehouse to an exhaust channel.

According to this configuration, even in the case in which a container storage rack to which inert gas is supplied by the inert gas supply device is inside a warehouse, use of the exhaust fan makes it possible to discharge inert gas from the inside of the warehouse to the exhaust channel. Accordingly, inert gas can be prevented from remaining inside the warehouse, and it is easy to reduce the inert gas concentration inside the warehouse.

Furthermore, it is preferable that the controller executes deceleration control to decelerate the airflow generated by the exhaust fan or deceleration prompting control to prompt a worker to perform an operation for decelerating the airflow generated by the exhaust fan, in response to the oxygen concentrations in all of the plurality of monitoring areas being higher than or equal to a deceleration threshold that is higher than or equal to the determination threshold.

According to this configuration, the airflow generated by the exhaust fan can be decelerated in a state in which there is no problem in the oxygen concentration, and thus the energy loss to drive the exhaust fan can be reduced.

The technique according to the present disclosure can be used for a container storage facility including a container storage rack with a plurality of container placement sections on which containers are respectively placeable, and an inert gas supply device configured to supply inert gas to each of the containers placed on the container placement sections.

What is claimed is:

1. A container storage facility comprising a container storage rack with a plurality of container placement sections on which containers are respectively placeable, and an inert gas supply device configured to supply inert gas to each of the containers placed on the container placement sections, the container storage facility-further comprising:
a sensor group constituted by a plurality of oxygen concentration sensors arranged in a distributed manner around the container storage rack;
a diffusion fan capable of blowing air to an intended point in the container storage rack; and
a controller,
wherein the controller divides an entire area of the container storage rack into a plurality of monitoring areas, estimates respective oxygen concentrations in the plurality of monitoring areas based on respective detection values of the plurality of oxygen concentration sensors constituting the sensor group, and, in response to any of the monitoring areas being a low oxygen concentration area in which the estimated oxygen concentration is lower than or equal to a predetermined determination threshold, operates the diffusion fan in such a manner as to blow air to the low oxygen concentration area.

2. The container storage facility according to claim 1,
wherein the sensor group comprises two or more of the oxygen concentration sensors arranged in a distributed manner at intervals in each of a first direction, a second direction, and a third direction, the first direction being a specific direction along a horizontal direction, the second direction being a direction orthogonal to the first direction in a vertical view along a vertical direction, and the third direction being a direction along the vertical direction, and
the controller estimates smallest values of respective oxygen concentrations in the plurality of monitoring areas with use of spatial interpolation based on detection values of all of the oxygen concentration sensors constituting the sensor group, and determines a monitoring area whose smallest value is lower than or equal to the determination threshold, as the low oxygen concentration area.

3. The container storage facility according to claim 1,
wherein the diffusion fan is configured to be capable of changing an airflow direction in response to a command from the controller.

4. The container storage facility according to claim 1, further comprising a display device,
wherein the controller displays, on the display device, an oxygen concentration map in which oxygen concentrations respectively estimated for the plurality of monitoring areas are associated with positions in the container storage rack.

5. The container storage facility according to claim 1,
wherein the container storage rack is inside a warehouse comprising a perimeter wall surrounding the container storage rack,
the perimeter wall comprises a portion with an opening, and
the diffusion fan generates an airflow in a direction intersecting a direction from an inside to an outside of the opening, in an area adjacent to the opening inside the warehouse.

6. The container storage facility according to claim 1,
wherein the container storage rack is inside a warehouse comprising a perimeter wall surrounding the container storage rack, and
the container storage facility further comprises an exhaust fan configured to generate an airflow from an inside of the warehouse to an exhaust channel.

7. The container storage facility according to claim 6,
wherein the controller executes deceleration control to decelerate the airflow generated by the exhaust fan or deceleration prompting control to prompt a worker to perform an operation for decelerating the airflow generated by the exhaust fan, in response to the oxygen concentrations in all of the plurality of monitoring areas being higher than or equal to a deceleration threshold that is higher than or equal to the determination threshold.
